(19) United States
(12) Patent Application Publication
(10) Pub. No.: US 2010/0316744 A1
(43) Pub. Date: Dec. 16, 2010

(54) PLANT SPROUT-BASED EMULSIONS, PROCESS FOR PREPARING THEM AND USE THEREOF

(76) Inventor: Ferenc Foglein, Budapest (HU)

Correspondence Address: BIRCH STEWART KOLASCH & BIRCH, PO BOX 747, FALLS CHURCH, VA, 22040-0747, US

(21) Appl. No.: 12/665,302

(22) PCT Filed: Jun. 17, 2008
(86) PCT No.: PCT/HU2008/000070

§ 371 Date: May 17, 2010

(30) Foreign Application Priority Data
Jun. 18, 2007 (HU) P0700421

(51) Int. Cl. A61K 36/736; A61K 36/00; A61K 36/28; A61K 36/31; A61K 36/48; A61K 36/185; A61K 36/87; A61K 36/52; A61K 36/55; A61P 3/04; A61Q 19/00; A23L 1/36

(52) U.S. Cl. 424/735; 424/776; 424/764; 424/755; 424/757; 424/766; 424/771; 424/768; 426/629

(57) ABSTRACT

The present invention relates to plant sprout-based stable oil-in-water emulsions wherein the monacyl- and diacylglycerol content is at least 20% by weight, the free fatty acid content is not more than 10% by weight and comprise an aqueous homogenate of sprouted oily seeds of which the own enzymes and emulsifying substances are capable to form the emulsion. Furthermore, the invention also relates to the preparation of said emulsions as well as to various applications thereof.

### FIELD OF THE INVENTION

The present invention relates to plant sprout-based stable oil-in-water emulsions wherein the monoacyl- and diacylglycerol content is at least 20% by weight, the free fatty acid content is not more than 10% by weight and comprise an aqueous homogenate of sprouted oily seeds of which the own enzymes and emulsifying substances are capable to form the emulsion. Furthermore, the invention also relates to the preparation of said emulsions as well as to various applications thereof.

### BACKGROUND OF THE INVENTION

Recent medical research suggests that high fat/lipid foodstuff and diets, particularly those high in cholesterol, saturated fatty acids and triglycerides, can contribute significantly to the development of many diseases, particularly heart diseases, atherosclerosis, high blood pressure and other cardiovascular diseases. In addition, obesity, often mentioned as endemic disease in many countries of the world, is also one of the risk factors of the above diseases.

Further, it is well known that oils contained in oily seeds are stored in the form of triglyceride and the oils subtracted therefrom with a cold or hot process (hot steam) are obtained in the form of triacylglycerol (TAG). The assimilation of thus obtained oils by the human digestive system is possible only if the secretion produced by the gall bladder previously forms an emulsion with the oils and fats. Failing this, lipases formed in the pancreas would be unable to degrade the oils. First, the bile secretion forms an emulsion, then the oils involved in the emulsion form micellae in an aqueous medium; as a consequence the fats become accessible to the water soluble lipases and degrade to form mono- and diacylglycerol as well as glycerol and free fatty acids.

It is also well known that the digestion of fats imposes a heavy duty upon the human digestive system resulting in an increasing demand for manufacturing such oils in which the lipids are present in a form accessible to lipases. Diacylglycerol (diglyceride, abbreviated as DAG) oils have proved to be highly appropriate for this purpose.

Diglyceride oils are generally described in numerous patent documents, e.g. in U.S. Pat. Nos. 5,160,759; 6,361,980 and 7,081,542, in Japanese published patent applications No. 63-301754, 5-168142 and 60180. Specifically, in U.S. Pat. No. 5,160,759 oil-in-water emulsions containing diglyceride oils are described, while U.S. Pat. No. 6,361,980 discloses the manufacturing of such diglycerides by means of an enzyme-based process. In U.S. Pat. No. 7,081,542 a chemical process is described for the production of 1,3-diglyceride oils wherein the alkali metal salts or alkali earth metal salts of mono-carboxylic or dicarboxylic acid are applied as catalyst in the glycerolysis. They are costly procedures, requiring highly sophisticated technical equipment. J. B. Kristensen et al. describe a cost saving experimental procedure for the preparation of DAG oil by lipase-catalyzed glycerolysis and by optimizing five parameters (Journal of Agricultural and Food Chemistry 53(18):7059-66, 2005).

WO 2005/048722 discloses drinks and bakery products containing DAG oil. For this, the DAG oil is prepared from fatty acids and glycerol by synthetic way and the DAG oil-in-water emulsions used in said products are prepared by the admixture of commercially available art-recognized emulsifiers. Such emulsions prepared with DAG oil show a high degree of emulsion stability. EP-A 1741342 discloses an oil or fat composition of high DAG content which contains plant sterol and plant sterol fatty acid ester.

Furthermore, it is also known that monoacyl-, diacyl- and triacylglycerols (MAG, DAG and TAG) possess emulsifying and stabilizing properties. Therefore, they are preferably applied in food industry products (see the Hungarian published patent application No. P9403335 and Hungarian patent Nos. 208066, 217528 and 217356), in pharmaceutical preparations, e.g. as vaccine adjuvants (Hungarian published patent application No. P0004001) or as emulsifiers for therapeutically active lipophylic substances (Hungarian published patent application No. P0002486 and Hungarian patent No. 225160), or for the introduction of biologically active substances into mammal organisms (Hungarian patent No. 220216), or for preparing oil-in-water type emulsions (Hungarian patent No. 221477).

In conclusion, the use of diglycerides in food products reduces the quantity of triglycerides in the blood, moreover, these molecules are primarily consumed for energy production and less of them will be deposited in the fatty tissues (Asia. Pac. J. Clin. Nutr. 16: 398-403, 2007).

In view of the above, a high need exists for more MAG and DAG oils as well as for the emulsions containing them. Considering that such oils are prepared till now in a chemical way or by the use of enzymes and the emulsions prepared from them contain some emulsifiers in most cases, the aim of the present invention is to prepare in a natural and profitable manner partly hydrolysed, easily digestible oil emulsions having low energy content.

It is generally appreciated that the germination process in sprouting seeds is triggered by the consumption of nutrients stored up. First, the necessary enzymes are induced or synthesized in order to mobilize the nutrient reserves that help the development of seedlings. In seeds of plants like flax, sunflower, rape, soy, etc., the predominant part of nutrient reserves is provided by oil (35-45%) and protein (15-20%). An important protease, amylase, cellulase and lipase activity can be detected during germination. A few such data with respect to flaxseed sprouts are mentioned in the international published patent application No. WO 03/003845. Our experiments support the fact that an important modification takes place in the protein structure of seeds under germination. Namely, with a 15-20% gradient polyacrylamide gel electrophoresis of the proteins, polypeptides and oligopeptides can be detected in an amount of 10-15% compared to the seeds being in unsprouted state.

Furthermore, it is known as well that the sprouts of a few of non-oily seeds have an emulsifying property.

In U.S. Pat. No. 5,958,473 carob seeds are treated with sulphuric acid at a high temperature, then neutralized. The sprout fraction of the carob seed is separated from the endosperm in a mechanical way (the seeds are not allowed to sprout) and the sprout fraction is treated at a high temperature in order to reduce its content in protease inhibitor. The high-protein content sprout fraction obtained this way is applied as emulsifier. An emulsion, for example mayonnaise, is prepared by the admixture of the oily and aqueous phases (see example 3 of U.S. Pat. No. 5,958,473).

In GB patent No. 2 356 790 such carob seed sprouts are used for stabilizing oil-in-water emulsions, that contain more than 25% by volume protease inhibitor. Carob seed sprouts have a good emulsifying property; the emulsions prepared with them retain their stability even if heat-treated. The above emulsion can be used as food component, for example in the processing of mayonnaise, sauces, soups, salad dressings, spreadable fats, desserts, milky desserts, ice-creams.

ES patent No. 8606781 describes a stable oil-in-water emulsion containing plant oil in which the oil phase comprises soy oil, maize germ and a caseinate or soy protein. In the article of the Journal of Food Science 57(3):726-731 (1992) the emulsifying capacity of soy proteins and of corn germ protein flour is studied.

In RU patent No. 2251890 it is described that the water-soluble proteins (leucosins) subtracted from wheat germ flour are applicable as emulsifiers for the manufacturing of food produces.

We have found during our experiments in an unpredictable way that oily seeds, such as flaxseeds, rapeseeds, sunflower seeds, etc. being homogenized and mixed up with water or physiological salt solution after germination, form an “oil-in-water emulsion” (hereinafter emulsion) which is stable and its components can not be separated from each other. This emulsion is of high mono- and diacylglycerol content, easily digested, miscible with water in any proportion, and does not separate to oil and water.

This detection is more surprising regarding the fact that the separation of the oil from the aqueous phase of the homogenate can easily be performed in case of homogenizing unsprouted flax-, rape- and sunflower seeds. Whereas in the case of sprouted seeds, it is impossible to separate the oil in a centrifugal way; namely, in each case an emulsion layer is obtained floating on top of the aqueous phase. This is, as mentioned earlier, a partly hydrolysed oil emulsion from which the oil does not separate even if allowed to stand.

The emulsion according to the present invention can be utilized either as an independent product or as an additive (e.g. as food additive) in food, pharmaceutical and beauty-care industry or in the field of feeding. On the basis of the experimental data, easy digestibility seems to be explained by the fact that oily seeds possess the enzymes and emulsifying substances necessary for utilizing their oil reserves, which substances, put together, behave like the bile liquid excreted by human organism.

Additionally, we have surprisingly found that the emulsion formed this way has a self-emulsifying property and is capable of emulsifying further adjuvant surplus quantities of the oil deriving from the seeds of the source plant or oils and fats coming from other sources. Regarding this advantageous property, it is suitable for the preparation of edible emulsions having low energy content. The resulting emulsion possesses a high degree of MAG and DAG content, while its TAG and free fatty acid content is significantly reduced compared with the TAG content of the adjuvant oil (see Tables 3 and 4).

### SUMMARY OF THE INVENTION

On the basis of the above, the present invention relates to a plant sprout-based stable oil-in-water emulsion wherein the monoacyl- and diacylglycerol content is at least 20% by weight, the free fatty acid content is not more than 10% by weight and comprises an aqueous homogenate of sprouted oily seeds of which the own enzymes and emulsifying substances are capable to form the emulsion.

The MAG and DAG content of the emulsion according to the present invention is preferably 40-80% and the free fatty acid content is about 1-8%. More preferably, the MAG and DAG content is 60-80% and the free fatty acid content is merely 1-5%.

For the purpose of the present invention, all sprouted seeds may be considered as starting material whose oil content is greater than 5% by weight. Preferable oily seeds for the sprouting are the seeds of sunflower, rape, flax, soy, sesame, hemp, maize, walnut, hazelnut, groundnut, almond, grape, black current and other seeds having said oil content.

Especially preferred are the demucilaged flax sprouts that can be obtained by sprouting flaxseeds deprived from mucilage as disclosed in the Hungarian published patent application No. P0500762. By depriving the flaxseeds from mucilage and utilizing the demucilaged flax sprouts, the possibility is open for both the active ingredients such as essential fatty acids, fito-estrogens and for the nutriments (proteins, carbohydrates, oils) contained in the flaxseeds to become accessible. Additionally, the demucilaged flax sprouts are an excellent basic material because their drying is performed in a manner that the active enzymes and vitamins remain unaffected; besides, the antioxidants generated during the germination of the flaxseeds provide protection for the product against oxidation.

Another preferred oily seed for the purpose of the present invention is sunflower seed. Mechanic hulling and the photoelectric sorting of the unhurt machine-hulled seeds enable the large-scale sprouting of sunflower seeds. Other seeds that can be envisaged for sprouting are the seeds of rape, sesame, grape, soy, maize, walnut, almond, hazelnut, black current and other seeds with oil content higher than 5%.

### DETAILED DESCRIPTION OF THE INVENTION

The invention is presented in more details through demucilaged flax sprouts. Fresh and dried demucilaged flax sprouts are prepared as described in the published Hungarian patent application No. P0500762.

For comparison, we have examined the lipid and fatty acid composition of cold-pressed flax sprout oil in the first 24 hours of germination by using gas chromatographic and thin layer chromatographic analyses (see Table 1).

TABLE 1
Change of the fatty acid and lipid composition of the demucilaged
flaxseed sprout oil during the first 24 hours of germination

| | Fatty acid % of samples | | | | |
|---|---|---|---|---|---|
| | control | after | after | after | after |
| Fatty acids | seed | 6 hours | 12 hours | 18 hours | 24 hours |
| Palmitic acid | 6.2 | 6.2 | 6.3 | 6.4 | 6.5 |
| (C 16:0) | | | | | |
| Palmitoleic | 0.1 | 0.1 | 0.1 | 0.1 | 0.1 |
| acid (C 16:1) | | | | | |
| Stearic acid | 6.2 | 6.4 | 6.5 | 6.5 | 6.6 |
| (C 18:0) | | | | | |
| Oleic acid | 22.2 | 21.8 | 21.4 | 20.9 | 20.8 |
| (C 18:1) | | | | | |
| Linoleic acid | 15.4 | 15.6 | 15.8 | 16.0 | 16.0 |
| (C 18:2) | | | | | |
| Linolenic acid | 52.2 | 52.1 | 51.2 | 51.0 | 51.0 |
| (C 18:3) | | | | | |
| Hydrolysed lipid | 0.5 | 5.0 | 8.2 | 17.5 | 24.3 |
| % DAG + MAG | | | | | |
| total | | | | | |

The data of Table 1 clearly show that the oil exploration is gradual when the sprout tissues are unhurt (i.e. were not previously homogenized). The total quantity of monoglyceride and diglyceride (MAG, DAG) in the extracted oil gradually increases until it reaches the value of 24.03%, while the ratio of saturated and unsaturated fatty acids as compared to each other and the total content of fatty acids do not significantly change.

In contrast to the above, when the freshly sprouted demucilaged flaxseeds are homogenized at ambient temperature with water or with physiological salt solution, the active enzymes act without any disturbance. By centrifuging the homogenate at 5000 rpm for 10 minutes, the oil quantity of about 45% contained in the flaxseeds (according to our own experiments) is entirely transformed to emulsion. If this emulsion is separated from the precipitate, it remains stable; moreover, it will not be fractionalized during storage. If the emulsion obtained in this way is diluted with water in a 1:10 ratio and is centrifuged again at 5000 rpm for 10 minutes, the emulsion may be washed out; it is stable, the oil and water do not separate from each other, only the oil-in-water emulsion can be separated from the washing water. This is very advantageous, because the eventual unwanted flavours can be removed this way. Furthermore, it was found that, during the process, an water-in-oil emulsion may be generated up to the rate of 5% by weight depending on the circumstances. On the given circumstances said emulsion can be transformed into oil-in-water emulsion as well.

We have examined the lipid composition of the stable oil-in-water emulsion with the TLC method (see Table 2).

TABLE 2
Degree of hydrolyzedness of the aqueous homogenate
of flax sprouts
% of lipids (determined by TLC)

| | | Total of | |
|---|---|---|---|
| Triacylglycerol | Total of diacyl- | monoacylglycerols | |
| (TAG) | glycerols (DAG) | (MAG) | Free fatty acids |
| 37 | 34 | 26 | 3 |

Table 2 demonstrates that flaxseed homogenate hydrolyses in an amount of about 60% of the oil into mono- and diacylglycerol in contrast to the value of 24.3 given in Table 1. Meanwhile, the value of free fatty acids does not increase.

Furthermore, when an oil derived from a plant species other than flax (e.g. sunflower- or rapeseed oil) was added to the flax sprout homogenate, it was found that the homogenate transformed this oil to a stable emulsion, regardless of the plant species the additional oil had derived from. Similar experiments were performed with the sprouting and homogenising of seeds of huskless sunflower, rape and soy. In every case we found that the homogenate took into a stable emulsion both its own oil and any other oil derived from different plant species. However, particularly preferred are the flax sprouts, because their homogenate contains an adequate quantity of antioxidant, thus the produce prepared in the above way does not change its colour and can be stored for a long time without becoming rancid. In this way, sunflower oil emulsion being more liable to rancidification, can be stored for a longer period. In addition, flax sprouts are also preferred because they contain essential fatty acids, such as omega-3 fatty acid, in a high quantity (see Table 6).

Besides, when fats originating from plants or animals were added to the emulsion according to the present invention, it was found that the emulsion hydrolysed in part and took into the emulsion the additional fat. Suitable fats for this purpose are palm-nut oil, butterfat, pork fat, beef tallow and the like. By adding fats to the emulsion, it may be hardened, but, when humans consumed it, the fats being partly hydrolysed will not be accumulated in the fatty tissues of the organism similarly to DAG-oils.

Furthermore, the invention relates to a plant sprout-based stable oil-in-water emulsion that contains also some additional oil and/or fat in an emulsified form. The additional oil may be either identical with the oil of the sprouted plant or may be an oil deriving from a different plant. The additional fat may be a vegetal and/or animal fat.

The invention further relates to a process for the preparation of the above plant sprout-based stable oil-in-water emulsions comprising the following steps:
(a) oily seeds are sprouted to obtain seedlings, then
(b1) the fresh seedlings are homogenised with water or with physiological salt solution; or
(b2) the grist of the dried seedlings are suspended in water or in physiological salt solution; or
(b3) the flour made from dried and oil-depleted seedlings is suspended in water or in physiological salt solution and a plant oil is added; or
(b4) a cleaned, fresh or dried isolate of sprout protein prepared from the fresh seedlings is diluted in water or in physiological salt solution and an oil is added;
(c) optionally, further oil and/or fat is added under mixing, and the thus-obtained stable emulsion is separated.

As used herein, the expression “fresh seedlings” means freshly sprouted seedlings with 24-28 hours of sprouting time. It should be understood that the “seedling” always comprises the seed under germination.

As used herein, the expression “grist of dried seedlings” means seedlings ground after they were gently dried at 35-42° C., preferably at 38° C.

As used herein, the expression “flour made from dried and oil-depleted seedlings” means dried and finely milled sprout-flour, from which the oil had been removed by cold pressing.

Under the expression “cleaned, fresh or dried isolate of sprout protein”, the isolate prepared according to the following process is understood. The unsoluble cell components and the oil-in-water emulsion are removed by centrifugation of the homogenate of the fresh flax sprouts prepared with water or physiological salt solution. The aqueous phase cleaned by centrifuging is separated and utilized fresh or after being gently dried (at 25-42° C.). The proteins are extracted from the cleaned aqueous phase in a manner known per se by precipitating them at the isoelectric point (pH=3) and then they can be dried. 1 g of the protein cleaned this way is capable of partly hydrolysing and introducing into the emulsion 50 g of oil.

The expressions “flaxseed sprout” and “flax sprout” and the ones “oil-in-water emulsion” and “emulsion” are used in the specification with identical meaning.

As used herein, the expression “aqueous homogenate” means a homogenate prepared with water or with physiological salt solution.

In one of the preferred embodiments of the process according to the present invention fresh seedlings are homogenized.

In another preferred embodiment of the process according to the present invention the grist of the dried seedlings is used. Physiological salt solution is added to the flax sprout grist previously dried in a mild manner, e.g. at 35° C. and it is dissolved while gently mixed. After the dissolution, it is centrifuged, whereupon the supernatant oily emulsion layer will contain the approximately total quantity of oil of the sprout grist. Alternatively, the dried flax sprout grist may be dissolved and simultaneously homogenized. In this case, the homogenate behaves similarly to the homogenate derived from fresh seedlings, i.e. it is capable of bringing into the emulsion a great quantity of the additional oil. This supports that the enzymatic system and the emulsion-forming substances of the dried flaxseed grist are active and operable.

In a further preferred embodiment of the process according to the present invention a flour prepared from dried and oil-depleted seedlings is used. Applying this version of the process, it is necessary to add a plant oil to the aqueous homogenate in order to form the emulsion. The added oil may derive from the starting oily seed or from another oily seed.

In a still further embodiment of the process according to the present invention the starting material is a cleaned, fresh isolate of sprout protein. It may be prepared as described above. An oil extracted by cold pressing from unsprouted flaxseeds is added to the separated aqueous phase cleaned by centrifugation, which may be considered as a cleaned protein solution. The stirring of the mixture is continued, then it is centrifuged, while the aqueous protein isolate and the added oil result in a stable emulsion. If the protein isolate is dried, first it is suspended in water or in physiological salt solution and the oil is added thereafter. 1 g of the dried protein isolate is capable of partly hydrolysing and bringing into the emulsion 50 g of oil.

The emulsion forming capacity of the aqueous homogenate of the fresh flax sprouts was also studied. For this purpose, 300 g of additional oil is added to 100 g of fresh flax sprout homogenate, for example cold-pressed flaxseed oil, it is strongly mixed for ten minutes, then the cell components insoluble in water are removed by centrifugation. It was found that a clean non-emulsified oil layer appeared above the emulsion layer. The aqueous and the oil containing layers are mixed again and allowed to stand for 24 hours at 38° C. By next day, a thick emulsion resulted, from which it is no longer possible to separate the oil fraction. We suppose that the homogenate hydrolysed up the free oils. From this experiment we concluded that the emulsion-forming and hydrolysing capacity of the flax sprout homogenate was in correlation with the time factor (see Table 3).

TABLE 3
Hydrolysis of linseed oil in aqueous homogenate of flax
sprout at 38° C. in 24 hours (TLC)

| Triacylglycerols | Diacylglycerols | Monoacylglycerols | Free fatty acids |
|---|---|---|---|
| 19% | 39% | 32% | 10% |

Table 3 shows that the flax sprout homogenate is capable of hydrolysing and introducing into the emulsion 81% of the triglyceride content of the additional linseed oil in the form of DAG (39%), MAG (32%) and free fatty acids (10%).

We have also examined the emulsion-forming capacity of the sunflower sprout aqueous homogenate (see Table 4). This experiment was performed at ambient temperature for about 1 hour.

TABLE 4
Hydrolysis of linseed oil in aqueous homogenate of
sunflower sprout at ambient temperature in 1 hour (TLC)

| Triacylglycerols | Diacylglycerols | Monoacylglycerols | Free fatty acids |
|---|---|---|---|
| 44% | 26.6% | 22% | 7.4% |

Table 4 shows that the aqueous homogenate of the sprouted sunflower seeds is also capable of hydrolysing 56% of the triglycerides (TAG) in the form of DAG (26.6%), MAG (22%) and free fatty acids (7.4%).

Furthermore, we have studied the water-absorption capacity of the oil-in-water emulsion according to the present invention. Our experiments show that the emulsion prepared according to the process of the present invention derived from flax sprouts contains about 10% of water. Its particularly great advantage is that the water content can be controlled and set to a desired value, for example, it can be reduced to 1-2% by centrifuging. The emulsion can be diluted unlimitedly with water or with foodstuffs containing water, e.g. with milk or fruit juice. By increasing the water content of the emulsion to 30%, it can be heat-treated at 120° C. without any changes in its structure. If the emulsion is in a compact, dried form (water content 1-2%), it can be dried up. If dried in a vacuum owen at 80° C., a soft paste-like substance is obtained. This dried emulsion becomes emulsion again only if repeatedly homogenized with water. The protein content of the emulsion is about 0.7-1.2%. The emulsion can be heat-treated, e.g. at 80-95° C., and thereby it will have a longer shelf life.

The stable oil-in-water emulsion prepared according to the present invention can be utilized in many fields. Thus, primarily in food industry to manufacture health-care products, e.g. easily digestible oils to reduce obesity, further, as additive in manufacturing of low-energy foodstuffs favourably affecting the health condition, e.g. drinks, diary products such as milk and butter cream enriched with omega-3-fatty acid. In addition, it may be applied as food supplement, e.g. formulated in capsules in itself or together with a carrier. It can similarly be used as fodder supplement in feeding. Preferably, it can be added to the drinking water of animals since it can unlimitedly be diluted with water. Besides, it can be utilized in beauty-care industry, e.g. as emollient or as auxiliary material, since any additive applied in beauty-care industry for enhancing absorption and for extending shelf life can easily be driven into the emulsion.

The benefits of the invention could be summarized as follows.
The present invention provides a cost saving basic material, especially for food industry, that is a sprout-based stable oil-in-water emulsion which can be prepared at normal temperature and pressure in a natural way without any auxiliary material. This widely contributes to the profitable manufacturing of the produce.
The emulsion according to the invention is highly advantageous in respect of health protection on account of its high mono- and diglycerol content; it is a product easy to digest, low in energy content and contains all the active natural substances, such as essential fatty acids, vitamins, fitohormones and antioxidants originally present in the plant sprouts.
The emulsion according to the invention is stable, therefore it can be cleaned; its water content can be controlled and set to a desired value.
The emulsion according to the invention can be heat-treated at 80-95° C., even at 120° C. if it contains 30% water, thereby it can be stored for a long time.
The emulsion according to the invention can widely be applied as a cost saving basic material or additive in food-, health-care or beauty-care industry as well as in feeding.
   The percentage values given in the specification mean percent by weight, unless otherwise specified.
   We illustrate the invention by the following non-limiting examples.

### EXAMPLES

### Example 1

### Preparation of Emulsion from Fresh Demucilaged Flax Sprouts

100 g of fresh demucilaged flax sprouts (germinated for 32 hours) are placed in a mixer, then 1-20 times, preferably 10 times, the quantity of water or of physiological (0.9%) NaCl solution is added. The mixture is homogenized at a high rotor speed (at least 1500 rpm) for 10 minutes, until the seedlings become totally pasty. The homogenate is centrifuged in a sway-out head rotor (Sorvall RC5B, manufacturer: Sorwall, USA) at 5000×g for 10 minutes. The supernatant emulsion is removed from the centrifuge tubes with a spoon, after that it is mixed with NaCl solution in an amount which is identical with the volume of the above emulsion and finally it is centrifuged again. 100 g of aqueous sprout homogenate yields 35-40 ml stable emulsion. The DAG content of the emulsion yield is 34%, the MAG content is 26% and the free fatty acid content is 3% (see Table 2).

### Example 2

### Preparation of Emulsion by Using the Grist of Dried Demucilaged Flax Sprouts

50 g of dried flaxseed grist is homogenized in a mixer in 800 ml 0.9% NaCl solution for 20 minutes at 1500 rpm as described in Example 1. The mixture is allowed to stand for 60-90 minutes, then homogenized again. This way we ensure a better dissolution and suspension of the dried starting material. The suspension is centrifuged at 5000×g for 10 minutes; the supernatant is separated and collected. 40 ml stable oil-in-water emulsion is obtained by using 50 g of flax sprout grist. After storage for 14 days at 5° C. (without heat treatment), the emulsion remains stable and no oil fraction will be separated.

### Example 3

### Preparation of Emulsion from Dried and Oil-Depleted Sunflower Sprouts

Hulled sunflower seeds (Atomic species) are washed and sterilized on their surface with NaOCl solution for 20 minutes. Then, the seeds are allowed to sprout for 48 hours. The germination process is stopped with vacuum drying at 35° C. The sprouts are dried until 0.5% humidity content or 0.5 wa (water activity). After drying, 85% of the oil content is removed with the help of an oil press. The substance remaining in the press is grounded to form a fine, powder-like flour, this way is obtained the sunflower sprout flour.

50 g of sunflower sprout flour is placed in a mixer; 0.15 M NaCl solution is added and mixed at 200 rpm for 5 minutes. After a foam appears, 200 ml of cold pressed linseed oil is added and the mixture is homogenized for further 10 minutes at 1500 rpm. As a consequence of this homogenization, an uniform emulsion is obtained. The homogenate is centrifuged at 5000×g for 10 minutes in order to remove the water-insoluble cell components. The thick emulsion obtained will not be separated to oil and water even during storage. The DAG content of the emulsion is 26.6%, the MAG content is 22% and the free fatty acid content is 7.4% (see Table 4).

### Example 4

### Preparation of Cleaned Oil Emulsion from Fresh Demucilaged Flax Sprouts

A fine pulp is prepared by mixing 50 kg of fresh flax sprouts deprived of their mucilaginous materials in a fine-pulper homogenizer (in a cutter) in the presence of 200 litre of 0.15 M NaCl solution for 30 minutes, then the homogenate is further stirred for another 20 minutes in the cutter while adding further 300 litre of NaCl solution. By using a spiral pump, the homogenate is transferred to a three-phase, horizontal centrifuge of continuous operation (e.g. Flottweg tricanter, manufactured by Flottweg, Germany). The speed of the centrifuge is set between 500 and 2500 rpm, preferably to 1800 rpm, and the flow rate is set between 200 and 800 litre/hour, preferably to 500 litre/hour. By proceeding this way, the supernatant oil emulsion, the protein containing aqueous phase and the fibres insoluble in water are separated. The supernatant oil emulsion is collected in a tub and is diluted by adding 500 litres of water and intensively mixed again. The thin emulsion obtained this way is further cleaned with the help of a milk separator. The milk separator is operated at the fix speed of 5000 rpm. By setting the flow rate to 700 litre/hour, a thick emulsion is obtained. The density and water content of the emulsion is controlled by setting the flow rate. The lipid composition of the emulsion obtained in this process is shown in Table 5.

TABLE 5
Lipid composition of flax sprout oil emulsion in %
(determined by TLC)

| Triacylglycerols | Diacylglycerols | Monoacylglycerols | Free fatty acids |
|---|---|---|---|
| 32 | 36 | 31 | 1 |

Table 5 shows that the DAG+MAG content of the cleaned oil emulsion is extraordinarily high, 67% and its free fatty acid content is merely 1%.

In Table 6, the composition of the flax sprout emulsion is specified, applying Hungarian Standards.

TABLE 6
Composition of flax sprout oil emulsion
(applying Hungarian Standards)

| | |
|---|---|
| Acidity number | 1.8% |
| Raw protein | 0.7% |
| Raw fat | 80.5% |
| Palmitic acid (C 16:0) R % | 5.3% |
| Stearic acid (C 18:0) R % | 3.8% |
| Oleic acid (C 18:1) R % | 21.6% |
| Linoleic acid (C 18:2) R % | 13.1% |
| Linolenic acid (C 18:3) R % | 56.2% |
| Free fatty acid FFA | 0.9% |
| Water content | 10.4% |

R % = relative %

Beside the chemical composition, the cleaned flax oil emulsion obtained in the above process is also characterised by physical parameters. The measurements are performed by means of a LUMiSizer 6120-112 device (producer: LUM GmbH, Berlin, DE) on 870 nm wavelength in LUM test tube (2 mm, PC, Rect. Synthetic Cell (110-131xx). The data obtained according to the method called 255pr10s2000 rpm1lf25grd regarding the cleaned flax oil emulsion are as follows:

| | |
|---|---|
| Density: | 933 kg/m³ |
| Refractive index: | 1.482-1.478i |
| Medium particle size: | 771 nm |
| Medium sedimentation velocity: | 13.8988 μm/s |

### Example 5

### Preparation of Emulsion from Sprout Protein Isolate Use of Sunflower Sprout Homogenate for Emulsifying Other Oils

The aqueous phase prepared in the tricanter according to Example 4 is collected in a mixing container. 100 litre of a mixture comprising 70% cold pressed sunflower oil (Atomic species) and 30% linseed oil is added to 100 litre of the above aqueous phase containing protein while intensively mixed. The mixture is stirred in a rotor mixer at a speed of 300 rpm for at least 2 hours at 30° C. The mixture is transformed to a milk-like thin emulsion, which is concentrated in a milk separator to the desired water content. Preferably, the flow rate is set to 700 litre/hour. By this process, an oil preparation can be obtained with a fatty acid composition ideal for human consumption (omega-6/omega-3 fatty acid=1:1).

### Example 6

The emulsion prepared according to Examples 1-5 is dried in a microwave vacuum drier, while heat treatment is also performed.

2 litres of emulsion is placed in a rotating disk microwave vacuum drier. The drying temperature is set to 80° C. Drying is performed under 560 mbar in order to avoid an explosion-like leaving of the water. 1700-1800 g of creamy substance is obtained after drying.

600 ml of water is added to 200 g of dried emulsion and is homogenized in a mixer at 1500 rpm for 10 minutes. The concentration and density of the emulsion can be controlled as described in Examples 2 and 3.

### Example 7

### Preparation of Beverages

The emulsion prepared according to Examples 1-5 is applied for the preparation of beverages. For example, the emulsion prepared according to Example 5 is diluted to fourfold its quantity with water containing 5 g/litre sugar, 0.5 g/litre NaCl, then is flavoured with vanilla, poured to 250 ml bottles to be closed with caps and pasteurised at 80° C. It can be consumed as energy drink.

### Example 8

### Preparation of Milk Enriched with Omega-3 Fatty Acid

The fat content of milk is reduced to 0.5% in the course of processing. To replace the extracted butterfat, 2.5% by volume flax sprout emulsion, as prepared in Example 1, is added to the milk. The product is pasteurised prior to bottling. The flax sprout emulsion added to the milk in this form does not cause any change in the flavour of milk.

### Example 9

### Preparation of Butter Cream Enriched with Omega-3 Fatty Acid

The aqueous phase separated in the tricanter according to Example 4 is used for the preparation of butter cream. The aqueous phase is heated to 38° C. While continuously mixing a similarly warmed mixture of oils and fats is added. This mixture contains 10% of butterfat, 60% of sunflower oil (of the species Atomic, its oil containing 83% of oleic acid) and 30% of cold pressed linseed oil.

100 litres of the above oily mixture is added to 100 litres of the aqueous phase (protein solution) separated in the tricanter. The mixture is homogenized at 300 rpm for 30 minutes. It is allowed to stand for 120 minutes; then is homogenized again for 30 minutes. The homogenate is separated in a milk separator. The flow rate of the separator is set to 150 litre/hour in order to remove the greatest possible quantity of water. The homogenate is directly decanted to the final recipients and is cooled down to 10° C. An easily spreadable butter cream with high omega-3 fatty acid content is obtained.

### Example 10

### Preparation of a Regenerating Collagen Beauty Cream with a High Lignan Content

5 kg of fresh demucilaged flax sprouts is reduced to pulp in a mixer in 15 litres of 0.15M NaCl solution at 1500 rpm for 30 minutes. 20 litres of cold pressed linseed oil (or any other desired oil composition) is added after the homogenization process and is mixed in a mixing tub at 300 rpm for further 2-3 hours. The suspension obtained this manner introduces into the emulsion the whole liquid quantity. The insoluble fibres are removed in a Sorvall RC 5B centrifuge in angular rotor at 2500×g for 15 minutes. The emulsion can directly be used externally as a lignan-containing emollient. Any type of additives commonly used in the beauty-care industry that improve absorption or enhance storability, can be introduced.

### Example 11

### Preparation of Food Supplement

1.3 ml of the emulsion prepared according to Example 1 or 0.5-0.8 g of the emulsion dried according to Example 5 is filled to hard gelatine capsules (2 ml Capsugel).

1. A plant sprout-based stable oil-in-water emulsion wherein the monoacyl- and diacylglycerol content is at least 20% by weight, the free fatty acid content is not more than 10% by weight and comprises an aqueous homogenate of sprouted oily seeds of which the own enzymes and emulsifying substances are capable to form the emulsion.

2. The emulsion according to claim 1, which contains the aqueous homogenate of sprouted oily seeds having at least 5% of oil content.

3. The emulsion according to claim 2 which contains the aqueous homogenate of sprouted seeds of sunflower, demucilaged flax, rape, soy, black current, grape, sesame, walnut, hazelnut, peanut or almond.

4. The emulsion according to claim 3 which contains the aqueous homogenate of demucilaged sprouted flaxseeds.

5. The emulsion according to claim 3 which contains the aqueous homogenate of sprouted sunflower seeds.

6. The emulsion according to claim 1 which has a MAG- and DAG content of about 40-80% and a free fatty acid content of about 1-8%.

7. The demucilaged flax sprout-based emulsion according to claim 4 which has a MAG- and DAG content of 60-80% and a free fatty acid content of 1-5%.

8. The emulsion according to claim 1 which contains additional oils and/or fats introduced into the emulsion.

9. The emulsion according to claim 8 wherein the additional oil is identical with the oil of the sprouted seeds.

10. The emulsion according to claim 8 wherein the additional oil is different from the oil of the sprouted seeds.

11. The emulsion according to claim 7 wherein the additional fat is a fat of plant and/or animal origin.

12. A process for the preparation of a plant sprout-based stable oil-in-water emulsion according to claim 1 comprising the following steps:
(a) oily seeds are sprouted to obtain seedlings, then
(b1) the fresh seedlings are homogenised with water or with physiological salt solution; or
(b2) the grist of the dried seedlings are suspended in water or in physiological salt solution; or
(b3) the flour made from dried and oil-depleted seedlings is suspended in water or in physiological salt solution and a plant oil is added; or
(b4) a cleaned, fresh or dried isolate of sprout protein prepared from the fresh seedlings is diluted in water or in physiological salt solution and an oil is added;
(c) optionally, oils and/or fats are added under mixing, and the thus-obtained stable emulsion is separated.

13. The process according to claim 12 wherein the sprout protein isolate is prepared as follows: a homogenate is prepared with water or physiological salt solution from fresh seedlings of oily seeds; this homogenate is cleaned by removing the water-insoluble cell components and the oil-in-water emulsion by centrifugation; the proteins are precipitated from the cleaned aqueous phase; and, if required, it is dried.

14. The plant sprout-based stable oil-in-water emulsion according to claim 1 which is obtained by the process according to claim 12.

15. The use of the plant sprout-based stable oil-in-water emulsion according to claim 1 as emulsifier in the emulsification of plant oils and plant or animal fats.

16. The use of the plant sprout-based stable oil-in-water emulsion according to claim 1 as food supplement.

17. A food supplement comprising a plant sprout-based stable oil-in-water emulsion according to claim 1.

18. The plant sprout-based stable oil-in-water emulsion according to claim 1 for use in food industry.

19. The plant sprout-based stable oil-in-water emulsion according to claim 1 for use in health-care or beauty-care industry.

20. The plant sprout-based stable oil-in-water emulsion according to claim 1 for use as additive in feeding.

21. The dried plant sprout-based stable oil-in-water emulsion according to claim 1 which contains 1-2% of bound water.
